(19) United States
(12) Patent Application Publication
(10) Pub. No.: US 2020/0168330 A1
(43) Pub. Date: May 28, 2020

(54) SYSTEM AND METHOD FOR AUTOMATED MULTIMODAL SUMMARIZATION IN CONTROLLED MEDICAL ENVIRONMENTS TO IMPROVE INFORMATION EXCHANGE FOR TELEMEDICINE

(71) Applicant: KONINKLIJKE PHILIPS N.V., Einhoven (NL)

(72) Inventors: Daniel Jason SCHULMAN, Jamaica Plain, MA (US); Grzegorz Andrzej TOPOREK, Cambridge, MA (US); Saman PARVANEH, Danvers, MA (US)

(21) Appl. No.: 16/692,791

(22) Filed: Nov. 22, 2019

Related U.S. Application Data
(60) Provisional application No. 62/771,307, filed on Nov. 26, 2018.

(51) Int. Cl. G16H 40/67; G10L 17/00; H04N 21/8549; A61B 5/117; G16H 10/60

(52) U.S. Cl. CPC G16H 40/67; G10L 17/005; A61B 5/0077; A61B 5/117; G16H 10/60; H04N 21/8549

(57) ABSTRACT

Various embodiments relate to a method and system for method for automated, multimodal summarization of a medical visit in a controlled medical environment, the method including the steps of capturing, by an audiovisual monitoring module and a plurality of medical devices, a data stream of the medical visit in the controlled medical environment, collecting, by the audiovisual monitoring module, a request for a telemedicine consultation, performing, by a summarization service module, a summary extraction to select portions of the data stream, generating, by the summarization service module, an audiovisual summary and transmitting, by the summarization service module, the audiovisual summary to a telemedicine provider.

### CROSS-REFERENCE TO PRIOR APPLICATIONS

This application claims the benefit of U.S. Provisional Application No. 62/771,307, filed on 26 Nov. 2018. This application is hereby incorporated by reference herein.

### TECHNICAL FIELD

This disclosure relates generally to telemedicine, and more specifically, but not exclusively, to providing a summary of a visit to a a clincian who conducts a telemedicine session by remotely evaluating clinical records.

### BACKGROUND

Virtual consultations with physicians (i.e., telemedicine consultations) between a patient and a clinician (distinguished from clinician-clinician interaction without direct patient involvement) are a growing approach to providing medical services including diagnosis, medical decision making, and mental health counselling.

Telemedicine consultations between a patient and a clinician may be either a standalone clinical encounter or may be augmenting a clinical visit, for example, by providing immediate access to a medical specialist or other clinician with medical expertise not available at the on-site location during a primary care visit.

Currently, there is a need for convenient, low-acuity healthcare delivery. Patients underutilize low-acuity care due to possible inconvenience, cost, insufficient care network availability, or high-risk conditions (e.g., COPD) that may make travel difficult and inconvenient.

This need is apparent due to the overutilization of high-acuity care (e.g., emergency rooms). For example, in 2015, of 130 million emergency department visits in the United States, approximately 70% of those emergency room visits did not require immediate emergency services.

Alternatively care models are growing, for example, retail clinics such as walk-in clinics located in retail stores, supermarkets, or pharmacies that provide limited services (e.g., vaccination, screening, minor illness treatment), and are staffed by, at most, a single nurse practitioner. Retail clinics and other alternative care delivery models are expected to have fewer clinical staff and lower-expertise staff on-site, as compared to traditional care delivery models.

### SUMMARY

A brief summary of various embodiments is presented below. Embodiments address a system and method for automated multimodal summarization in controlled medical environments to improve information exchange for telemedicine.

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments described herein relate to a method for automated, multimodal summarization of a medical visit in a controlled medical environment, the method including the steps of capturing, by an audiovisual monitoring module and a plurality of medical devices, a data stream of the medical visit in the controlled medical environment, collecting, by the audiovisual monitoring module, a request for a telemedicine consultation, performing, by a summarization service module, a summary extraction to select portions of the data stream, generating, by the summarization service module, an audiovisual summary and transmitting, by the summarization service module, the audiovisual summary to a telemedicine provider.

In an embodiment of the present disclosure, the method for automated, multimodal summarization of a medical visit in a controlled medical environment, the method further including the steps of receiving, by the summarization service module, feedback from the telemedicine provider on the audiovisual summary and improving, by the summarization service module, the generation of the audiovisual summary using the feedback from the telemedicine provider.

In an embodiment of the present disclosure, the data stream is annotated with events before being transmitted to the summarization service module.

In an embodiment of the present disclosure, the controlled medical environment includes a patient identification module configured to identify an individual in the controlled medical environment and include position and identity in the data stream.

In an embodiment of the present disclosure, the controlled medical environment includes a text transcription module configured to transcribe speech from the audiovisual monitoring module into text and include the text in the data stream.

In an embodiment of the present disclosure, the controlled medical environment includes a voice transcription module configured to identify an individual's voice and transcribe the individual voice into text and include the text in the data stream.

In an embodiment of the present disclosure, the controlled medical environment includes a trigger module configured to detect a request for a telemedicine consultation by a direct request, an indirect request or fulfillment of a pre-specified trigger for a telemedicine consultation.

In an embodiment of the present disclosure, the summarization service module includes a summary extraction module configured to receive the events and the request for the telemedicine consultation and outputs a subset of events.

In an embodiment of the present disclosure, the summarization service module includes a summary presentation module configured to receive a video stream from the audiovisual monitoring module and the subset of events and assembles a summary video in a temporal order.

In an embodiment of the present disclosure, the summary presentation module in the summarization service module augments the summary video by captioning the summary video and providing a visual display of measurements from the plurality of medical devices.

Various embodiments described herein relate to a system for automated, multimodal summarization of a medical visit in a controlled medical environment, the system including an audiovisual monitoring module and a plurality of medical devices configured to capture a data stream of the medical visit in the controlled medical environment, wherein the audiovisual monitoring module is configured to collect a request for a telemedicine consultation; and a summarization service module configured to perform a summary extraction to select portions of the data stream, generate an audiovisual summary and transmit the audiovisual summary to a telemedicine provider.

In an embodiment of the present disclosure, the summarization service module is further configured to receive feedback from the telemedicine provider on the audiovisual summary and improve the generation of the audiovisual summary using the feedback from the telemedicine provider.

In an embodiment of the present disclosure, the data stream is annotated with events before being transmitted to the summarization service module.

In an embodiment of the present disclosure, the controlled medical environment includes a patient identification module configured to identify an individual in the controlled medical environment and include position and identity in the data stream.

In an embodiment of the present disclosure, the controlled medical environment includes a text transcription module configured to transcribe speech from the audiovisual monitoring module into text and include the text in the data stream.

In an embodiment of the present disclosure, the controlled medical environment includes a voice transcription module configured to identify an individual's voice and transcribe the individual voice into text and include the text in the data stream.

In an embodiment of the present disclosure, the controlled medical environment includes a trigger module configured to detect a request for a telemedicine consultation by a direct request, an indirect request or fulfillment of a pre-specified trigger for a telemedicine consultation.

In an embodiment of the present disclosure, the summarization service module includes a summary extraction module configured to receive the events and the request for the telemedicine consultation and outputs a subset of events.

In an embodiment of the present disclosure, the summarization service module includes a summary presentation module configured to receive a video stream from the audiovisual monitoring module and the subset of events and assembles a summary video in a temporal order.

In an embodiment of the present disclosure, the summary presentation module in the summarization service module augments the summary video by captioning the summary video and providing a visual display of measurements from the plurality of medical devices.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates a block diagram of the system for automated multimodal summarization in controlled medical environments to improve information exchange for telemedicine of the current embodiment;

FIG. 2 illustrates a block diagram for the controlled medical environment of the current embodiment;

FIG. 3 illustrates a flow diagram for the summarization service of the current embodiment; and

FIG. 4 illustrates a block diagram of a real-time data processing system of the current embodiment.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

An issue that arises with use of these alternative care models is the expertise available at the locations.

The current embodiments address this need by providing ad-hoc (i.e., on demand, without prior scheduling) telemedicine consultation as part of a medical visit as a solution to addressing a limited set of capabilities and expertise available in a retail clinic.

The current embodiments may also be applied to other environments, apart from traditional hospital facilities, in which patient data is gathered and exposed to a clinician for further evaluation. This may include a patient's home, ambulance, remote primary care offices, interventional suites, operating rooms, etc.

However, by using a telemedicine consultation, efficient and accurate information exchange when starting a telemedicine consultation becomes required when using this ad-hoc model.

A medical provider performing a telemedicine consultation with a patient (i.e., a telemedicine provider) acquires the required information on the patient, including a medical complaint and/or condition, and the events of the clinical visit prior to the start of an ad-hoc telemedicine session.

The telemedicine provider may be given information that was available prior to the start of the visit, for example, through the patient's electronic health record (“ERR”) and complaint made at the time of scheduling a visit. The telemedicine provider may ask the patient, during a medical interview for information, such as the events during the visit and the reason for a telemedicine consultation. However, this process may be time-consuming, which may reduce the efficiency and patient engagement and/or satisfaction with the telemedicine consultation and may risk patient errors or omissions. Alternatively, the telemedicine provider may ask an on-site provider, for example, the nurse practitioner, if available, however, this process will have a similar loss of time and may lead to loss of patient engagement and satisfaction with the telemedicine consultation, because medical providers (e.g., the nurse practitioner and telemedicine provider) are now interacting with each other rather than the patient).

An on-site provider (i.e., a nurse practitioner) may prepare medical notes for a telemedicine provider summarizing the patient's status and complaint, the events of the visit, and the reason for a telemedicine consultation. However, this is a similarly time-consuming process, causing a delay prior to the start of a telemedicine consultation which may lose efficiency in the process and may risk loss of patient engagement and satisfaction.

The current embodiments address the issues with the process of informing the telemedicine provider with the necessary details for the telemedicine consultation by providing a brief, automatically-generated summary of a visit complemented by measurements which were performed by the medical equipment that a telemedicine provider may review prior to the start of a telemedicine consultation, which will result in a minimal loss of efficiency or patient engagement. The telemedicine provider may retain the ability to conduct an interview with the patient to fill in gaps or confirm any information. The information exchanged when starting a telemedicine session (i.e. the automatically-generated summary) may be captured for retrospective examination, to allow for future improvement.

The current embodiment is a system which captures an audiovisual data stream of a clinical visit, annotated with events (e.g., marked by a nurse practitioner or other provider and/or automatically captured by medical device and clinical decision support tool), collects a “trigger” which is a request for a telemedicine session, performs a summary extraction to select portions of the data stream, generates an audiovisual summary, augmented with additional information (e.g., measurements from medical devices), delivers the audiovisual summary to a telemedicine provider and updates and, optionally and at a later time, improves a summarization service module using feedback by a telemedicine provider.

The medical devices may perform acquisition of medical measurements without supervision of the clinician, allowing the audiovisual data stream to be created at a patient's home, using, for example, a mobile phone camera with a special application, annotated with a computer aided design (“CAD”) system and sent for further evaluation.

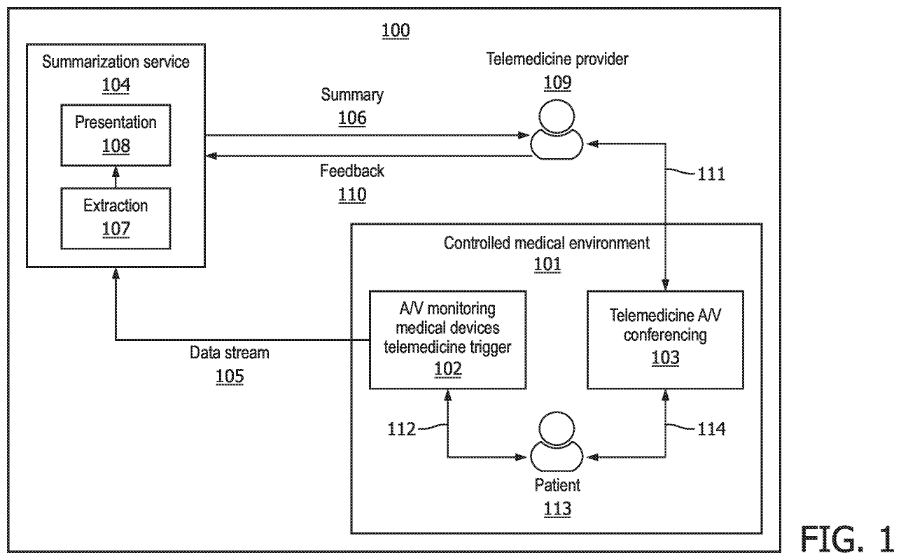

FIG. 1 illustrates a block diagram of the system 100 for automated multimodal summarization in controlled medical environments to improve information exchange for telemedicine of the current embodiment.

The system 100 includes a controlled medical environment module 101 which is an environment including a set of integrated medical devices 102 which are arranged in a known configuration and are capable of communicating through known protocols with each other and with additional modules and further are capable of audiovisual monitoring and network communication. A telemedicine audiovisual conferencing module 103 is used for a telemedicine consultation.

The patient 113 may provide information to the set of integrated medical devices 102 through path 112 and may provide information to the telemedicine provider 109 through path 114 and path 111.

The controlled medical environment module 101 captures an annotated audiovisual data stream and a trigger event using the set of integrated set of medical devices 102.

The system 100 further includes a summarization service module 104 that receives data from a data stream 105 from the set of integrated medical devices 102 in the controlled medical environment 101 and produces a summary 106 through a pipeline of modules including a summary extraction module 107 and a summary presentation module 108.

The controlled medical environment 101 may be available in primary-care office, patient home, traditional healthcare provider offices, interventional radiological suites or surgical operating rooms.

In an alternative embodiment, the summarization service module 104 may be physically located in the controlled medical environment 101, with the telemedicine provider 109, or separately from either, but in all cases, must be connected via an appropriate network and able to securely and reliably transmit an audiovisual data stream to both. The summarization service module 104 may utilize feedback 110 by a telemedicine provider 109 to improve report extraction and presentation.

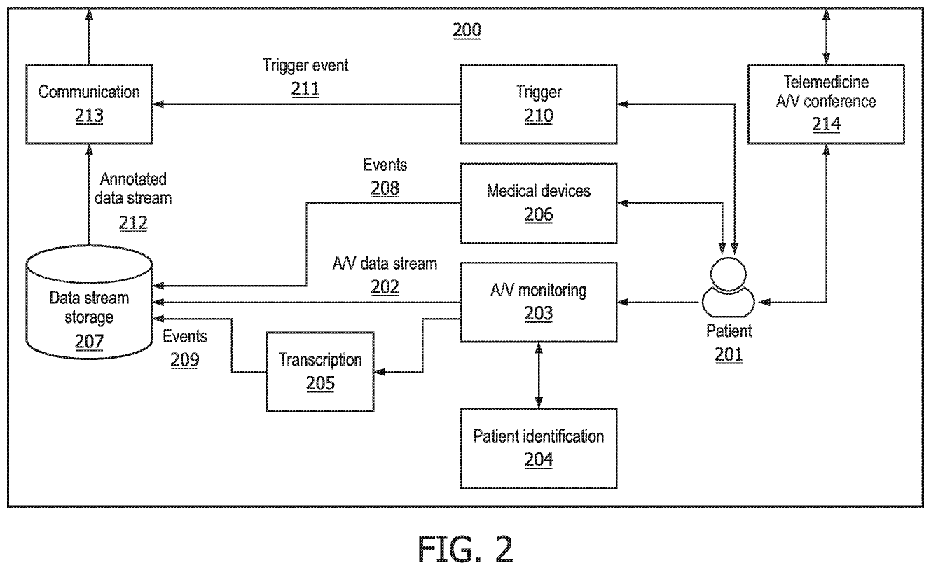

FIG. 2 illustrates a block diagram for the controlled medical environment 200 of the current embodiment.

The controlled medical environment 200 (from FIG. 1) includes an audiovisual monitoring system 203 capable of recording the medical visit, including the patient 201 at all times during a medical visit and any on-site provider, if present. In interventional X-ray rooms, in a controlled medical environment 200, the audiovisual monitoring system 203 may also incorporate medical staff (technicians, interventionalists, etc.) and the monitoring of medical staff may help in radiation dose management, fatigue reduction and improvement of the ergonomics.

The audiovisual monitoring module 203 may be implemented by standard methods and equipment and may be configured to produce a continuous audiovisual data stream 202 during a medical visit.

The audiovisual monitoring system 203 may include a monocular camera and a single microphone, two or more sets of stereoscopic cameras and two or set of calibrated microphones, a single or a set of depth cameras (e.g., time-of-flight cameras, etc.), LIDAR, video stream from the augmented reality headsets, other cameras (e.g. infrared cameras, hyperspectral cameras. Thermal cameras, etc.) and microphones, or a combination thereof.

The controlled medical environment module 200 may include a patient identification module 204 which may distinguish the position and identity of the patient 201, any medical provider (i.e., a nurse practitioner) and other individuals (e.g., a family member) in the controlled medical environment and output annotations for the data stream providing the position and identity. The patient identification module 204 may use a retina scanning and recognition devices, or simple password identification (PIN, patient ID, etc.), or vison-based face recognition system that may acquire a photo of the patient's face (or other individuals present during the examination) and match it with a large set of available photos in telemedicine provider's database. The matching of the photos may be achieved by using facial recognition algorithms, such as those based on convolutional neural networks. Images of the patient's face may be either acquired when the patient enters the examination room in the controlled medical environment 200 or during the medical visit using the audiovisual monitoring system 203. The patient identification module 204 may use a voice recognition system that may acquire a voice of the patient (or other individuals present during the examination) and match it with a large set of available voices in telemedicine provider's database. The matching of the voices may be achieved by using voice recognition algorithms, such as those based on convolutional neural networks. Voice of the patient may be either acquired when the patient enters the examination room in the controlled medical environment 200 or during the medical visit using the audiovisual monitoring system 203.

The controlled medical environment module 200 may include a text transcription module within the transcription module 205 which may apply speech-to-text technology, which may be trained on samples of speech from similar clinical visits and may output annotations for the audiovisual data stream 202. The text transcription module may be specialized with available information from the clinical record, e.g. the age and/or gender of the patient.

The controlled medical environment 200 may include a voice transcription module within the transcription module 205 which may apply voice recognition methods including deep recurrent or long short-term memory neural networks as well as Gaussian Mixture Models and may output text annotations and captions given from a visual data stream from the audiovisual monitoring system 203.

The controlled medical environment 200 may include a set of integrated medical devices 206 which may output events (e.g., vital signs and other measurements) as procedures are performed during the visit. Events may be timestamped and form additional annotations for the audiovisual data stream 202. Events may be marked by a nurse practitioner or other provider and/or automatically detected by a clinical decision support tool (e.g., automatic detection of lung wheezing).

The controlled medical environment may include a data stream storage device 207, which may receive events 208 from the integrated set of medical devices module 206, the audiovisual data stream 202 from the audiovisual monitoring module 203 and events 209 from the transcription module 205 from the audiovisual monitoring module 203 and may output an annotated data stream 212 to a communication module 213.

The controlled medical environment 200 may include a trigger module 210 which may either, for a semi-autonomous controlled medical environment 200, with an on-site clinician, accept a request, either as text or spoken and transcribe to text, for a telemedicine consultation from the on-site clinician or, for either a semi-autonomous or fully-autonomous controlled medical environment 200, automatically create a request in response to a user's verbal request, other user's speech indicating a need for a telemedicine consultation (e.g., a question outside the capabilities of the controlled medical environment 200) or an automatically detected event by the integrated set of medical devices 206 which may be pre-specified to trigger a telemedicine consultation (e.g., automatic detection of hypoxia).

Events that trigger 211 a telemedicine consultation may include a measurement by the integrated set of medical devices 206 outside of normal ranges, anomalies and undesired trends in events generated by the integrated set of medical devices 206, anomalies that are induced either by incorrect or disarrayed operation or system errors which may be detected from constantly monitored log data, abnormal behavior of the patient 201 (e.g., syncope, collapse or fainting of the patient).

The controlled medical environment 200 may include a communication module 213 which may transmit a data stream, annotated with events from modules 203, 204, 205, 206 and 210 (i.e., text transcription, voice transcription, log data and measurements from the integrated set of medical devices) and accompanied by a representation of the trigger event 211.

The controlled medical environment 200 may include a telemedicine audiovisual conferencing module 214 for performing the telemedicine consultation.

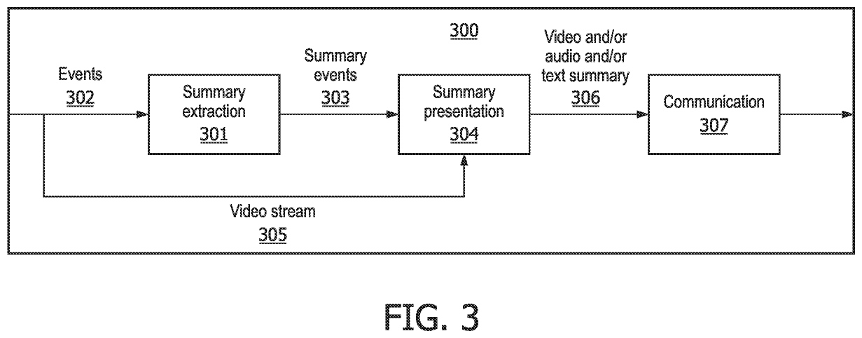

FIG. 3 illustrates a flow diagram for the summarization service 300 of the current embodiment.

The summarization service 300 includes a summary extraction module 301 which receives the sequence of timestamped events 302 (i.e., transcribed text and device measurements and other events from the controlled medical environment, each annotated with the time and duration of the corresponding segment of video) and the trigger event for the summary and outputs a chosen subset of the events 303 under specified constraints such as a maximum summary duration.

The summarization service 300 further includes a summary presentation module 304 which receives a video stream 305 and the subset of events 303 chosen by the summary extraction module 301 and assembles a summary video and/or audio and/or text 306 which includes segments of video/event streams corresponding to the chosen summary events, in a temporal order.

The summary presentation module 304 further augments this video stream with audiovisual representations of events, such as the captioning of transcribed speech and visual display of measurements from devices. Audiovisual representations of events may be overlaid on the video stream, displayed in juxtaposition with it, or shown by other methods.

The summarization service 300 further includes a communication module 307 which transmits the summary (i.e., a video stream) to the telemedicine provider for the telemedicine consultation.

The summary extraction module may be based on, for example, a maximum marginal relevance (“MMR”) method, however, others methods may be used, such as submodular function maximization methods, and recurrent neural network models The MMR method requires defining a measure of similarity between events, and a measure of event relevance or importance. In the current embodiment, similarity is defined as any convenient measure of text similarity (e.g., cosine distance) for textual events (e.g., transcriptions of speech) and by pre-specified similarities between different types of measurement events (e.g., measurements of the same vital signs are specified to be similar). Relevance may be defined as similarity to the trigger event. Therefore, a summary may be constructed by selecting the highest relevance event and repeatedly, until the maximum specified summary duration is reached, selecting the event with the highest marginal relevance, defined as the relevance of an event minus the maximum similarity to any previously selected event.

In the current embodiment, an additional voice transcription module may be used to analyze the conversation between the telemedicine provider and the patient and augment the telemedicine provider's screen with the additional conversation information.

The additional voice transcription module constantly monitors and transcribes, to readable text, a conversation between a telemedicine provider and a patient. The additional voice transcription module may apply voice recognition methods, based on either deep recurrent or long short-term memory neural networks or Gaussian Mixture Models, or other voice recognition methods. The additional voice transcription module may output text annotations and captions given a visual data stream from the telemedicine session.

When a similarity between the current and the previously summarized transcription is above a specified threshold, the system augments the telemedicine provider's panel with information that was previously captioned with detected word or expression and because conversation may contain either noisy or partially related information, similarity between words or sentences is calculated using deep neural networks or other machine learning models. For example, using Siamese recurrent neural networks for natural language processing for learning semantic sentence similarity.

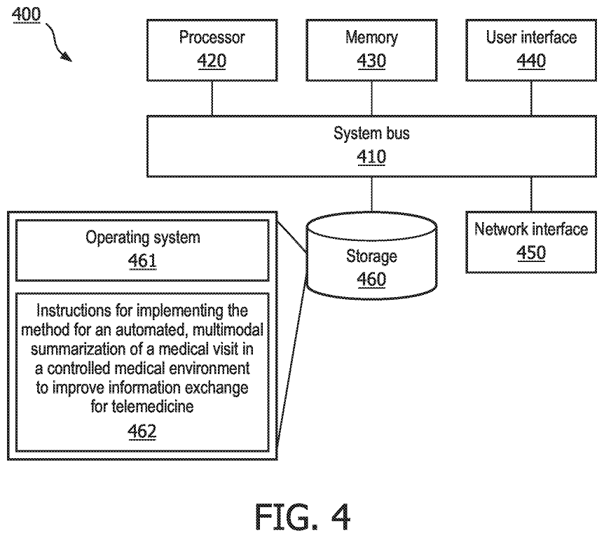

FIG. 4 illustrates an exemplary hardware diagram 400 for implementing a method for hybrid trust management for health records audit. As shown, the device 400 includes a processor 420, memory 430, user interface 440, network interface 450, and storage 460 interconnected via one or more system buses 410. It will be understood that FIG. 1 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, field programmable gate array (FPGA), application-specific integrated circuit (ASIC), or other similar devices.

The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices.

The user interface 440 may include one or more devices for enabling communication with a user such as an administrator. For example, the user interface 440 may include a display, a mouse, and a keyboard for receiving user commands. In some embodiments, the user interface 440 may include a command line interface or graphical user interface that may be presented to a remote terminal via the network interface 450.

The network interface 450 may include one or more devices for enabling communication with other hardware devices. For example, the network interface 450 may include a network interface card (NIC) configured to communicate according to the Ethernet protocol. Additionally, the network interface 450 may implement a TCP/IP stack for communication according to the TCP/IP protocols. Various alternative or additional hardware or configurations for the network interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon with the processor 420 may operate. For example, the storage 460 may store a base operating system 461 for controlling various basic operations of the hardware 400 and instructions for an automated, multimodal summarization of a medical visit in a controlled medical environment to improve information exchange for telemedicine 462.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered “non-transitory machine-readable media.” As used herein, the term “non-transitory” will be understood to exclude transitory signals but to include all forms of storage, including both volatile and non-volatile memories.

While the host device 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Further, where the device 400 is implemented in a cloud computing system, the various hardware components may belong to separate physical systems. For example, the processor 420 may include a first processor in a first server and a second processor in a second server.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method for automated, multimodal summarization of a medical visit in a controlled medical environment, the method comprising the steps of:
capturing, by an audiovisual monitoring module and a plurality of medical devices, a data stream of the medical visit in the controlled medical environment;
collecting, by the audiovisual monitoring module, a request for a telemedicine consultation;
performing, by a summarization service module, a summary extraction to select portions of the data stream;
generating, by the summarization service module, an audiovisual summary; and
transmitting, by the summarization service module, the audiovisual summary to a telemedicine provider.

2. The method for automated, multimodal summarization of a medical visit in a controlled medical environment of claim 1, the method further comprising the steps of:
receiving, by the summarization service module, feedback from the telemedicine provider on the audiovisual summary; and
improving, by the summarization service module, the generation of the audiovisual summary using the feedback from the telemedicine provider.

3. The method for automated, multimodal summarization of a medical visit in a controlled medical environment of claim 1, wherein the data stream is annotated with events before being transmitted to the summarization service module.

4. The method for automated, multimodal summarization of a medical visit in a controlled medical environment of claim 1, wherein the controlled medical environment includes a patient identification module configured to identify an individual in the controlled medical environment and include position and identity in the data stream.

5. The method for automated, multimodal summarization of a medical visit in a controlled medical environment of claim 1, wherein the controlled medical environment includes a text transcription module configured to transcribe speech from the audiovisual monitoring module into text and include the text in the data stream.

6. The method for automated, multimodal summarization of a medical visit in a controlled medical environment of claim 1, wherein the controlled medical environment includes a voice transcription module configured to identify an individual's voice and transcribe the individual voice into text and include the text in the data stream.

7. The method for automated, multimodal summarization of a medical visit in a controlled medical environment of claim 1, wherein the controlled medical environment includes a trigger module configured to detect a request for a telemedicine consultation by a direct request, an indirect request or fulfillment of a pre-specified trigger for a telemedicine consultation.

8. The method for automated, multimodal summarization of a medical visit in a controlled medical environment of claim 3, wherein the summarization service module includes a summary extraction module configured to receive the events and the request for the telemedicine consultation and outputs a subset of events.

9. The method for automated, multimodal summarization of a medical visit in a controlled medical environment of claim 8, wherein the summarization service module includes a summary presentation module configured to receive a video stream from the audiovisual monitoring module and the subset of events and assembles a summary video in a temporal order.

10. The method for automated, multimodal summarization of a medical visit in a controlled medical environment of claim 9, wherein the summary presentation module in the summarization service module augments the summary video by captioning the summary video and providing a visual display of measurements from the plurality of medical devices.

11. A system for automated, multimodal summarization of a medical visit in a controlled medical environment, the system comprising:
an audiovisual monitoring module and a plurality of medical devices configured to capture a data stream of the medical visit in the controlled medical environment, wherein the audiovisual monitoring module is configured to collect a request for a telemedicine consultation; and
a summarization service module configured to:
perform a summary extraction to select portions of the data stream;
generate an audiovisual summary; and
transmit the audiovisual summary to a telemedicine provider.

12. The system for automated, multimodal summarization of a medical visit in a controlled medical environment of claim 11, wherein the summarization service module is further configured to receive feedback from the telemedicine provider on the audiovisual summary and improve the generation of the audiovisual summary using the feedback from the telemedicine provider.

13. The system for automated, multimodal summarization of a medical visit in a controlled medical environment of claim 11, wherein the data stream is annotated with events before being transmitted to the summarization service module.

14. The system for automated, multimodal summarization of a medical visit in a controlled medical environment of claim 11, wherein the controlled medical environment includes a patient identification module configured to identify an individual in the controlled medical environment and include position and identity in the data stream.

15. The system for automated, multimodal summarization of a medical visit in a controlled medical environment of claim 11, wherein the controlled medical environment includes a text transcription module configured to transcribe speech from the audiovisual monitoring module into text and include the text in the data stream.

16. The system for automated, multimodal summarization of a medical visit in a controlled medical environment of claim 11, wherein the controlled medical environment includes a voice transcription module configured to identify an individual's voice and transcribe the individual voice into text and include the text in the data stream.

17. The system for automated, multimodal summarization of a medical visit in a controlled medical environment of claim 11, wherein the controlled medical environment includes a trigger module configured to detect a request for a telemedicine consultation by a direct request, an indirect request or fulfillment of a pre-specified trigger for a telemedicine consultation.

18. The system for automated, multimodal summarization of a medical visit in a controlled medical environment of claim 13, wherein the summarization service module includes a summary extraction module configured to receive the events and the request for the telemedicine consultation and outputs a subset of events.

19. The system for automated, multimodal summarization of a medical visit in a controlled medical environment of claim 18, wherein the summarization service module includes a summary presentation module configured to receive a video stream from the audiovisual monitoring module and the subset of events and assembles a summary video in a temporal order.

20. The system for automated, multimodal summarization of a medical visit in a controlled medical environment of claim 19, wherein the summary presentation module in the summarization service module augments the summary video by captioning the summary video and providing a visual display of measurements from the plurality of medical devices.
